(19) United States
(12) Patent Application Publication
(10) Pub. No.: US 2017/0035601 A1
(43) Pub. Date: Feb. 9, 2017

(54) ORAL DEVICE FOR TREATING SLEEP DISORDERED BREATHING DISORDERS

(71) Applicant: CATHOLIC UNIVERSITY INDUSTRY-ACADEMIC COOPERATION FOUNDATION, Seoul (KR)

(72) Inventor: Sang Hwa LEE, Seoul (KR)

(73) Assignee: CATHOLIC UNIVERSITY INDUSTRY-ACADEMIC COOPERATION FOUNDATION, Seoul (KR)

(21) Appl. No.: 15/304,234

(22) PCT Filed: Mar. 25, 2015
(86) PCT No.: PCT/KR2015/002910

§ 371 Date: Oct. 14, 2016

(30) Foreign Application Priority Data
Apr. 16, 2014 (KR) 10-2014-0045157

(51) Int. Cl. A61F 5/56

(52) U.S. Cl. CPC A61F 5/566

(57) ABSTRACT

The present invention relates to an oral device for treatment of sleep disordered breathing that includes an upper body in which at least a part of upper teeth is housed; a lower body in which at least a part of the lower teeth is housed; at least one or more elastic members that connect the upper body and the lower body to provide the elastic force in a direction in which the upper body and the lower body come closer to each other; and a support jaw that protrudes downward from the rear end of the upper body to support the rear end of the lower body. Thus, according to the present invention, it is possible to prevent the sleep disordered breathing such as an obstructive sleep apnea syndrome that may occur during sleep, enable a patient to more comfortably wear, and minimize the discomfort in a state of wearing.

### TECHNICAL FIELD

The present invention relates to an oral device for treatment of sleep disordered breathing, and more particularly, to an oral device for treatment of sleep disordered breathing which can prevent the sleep disordered breathing such as an obstructive sleep apnea syndrome that may occur during sleep, enables a patient to more comfortably wear, and can minimize the discomfort in a state of wearing.

### BACKGROUND ART

The sleep disordered breathing is a disease in which the patient suffers from the breathing disorder during sleep to worsen the health condition of the patient, shows symptoms of sleep disorders such as severe snoring and daytime sleepiness during sleep, and frequently causes breathing stop during sleep, which may cause a variety of complications cardiopulmonary vascular system by hypoxemia or the like.

Typical symptoms of such sleep disordered breathing, there is a snoring, obstructive sleep apnea syndrome or the like. The snoring is a phenomenon in which, while an interval between the pharyngeal wall and the surrounding soft tissue is narrowed during inspiration, such a narrowed part vibrates to generate a sound during inspiration, and an obstructive sleep apnea syndrome is a phenomenon in which as the pharyngeal wall and the soft tissue around the respiratory tract are excessively relaxed and the soft tissue becomes close contact with the pharyngeal wall, and the upper respiratory tract is narrowed, while the flow of air is partially blocked, the respiratory tract is temporarily blocked, and thus, air cannot temporarily flow into the lung.

Since patients suffering from the snoring and obstructive sleep apnea syndrome frequently wake up during sleep, a quality of sleep is lowered, chronic fatigue symptoms and chronic headache symptoms may be caused by repetitive hypoxia.

Oral devices of various shapes have been developed to treat patients exhibiting symptoms of sleep disordered breathing as described above. In general, as the oral devices, a calibration method of pushing a mandible (lower jaw) forward to open the respiratory tract during sleep has been used.

Although the lower jaw can be moved forward and fixed when worn or the lower jaw can move vertically in most of the conventional oral devices, there was a difficulty of movement in a horizontal direction. Therefore, the patient has felt the considerable discomfort during sleep after the wearing the oral device, which has also disturbed the sleep.

### DISCLOSURE

### Technical Problem

Accordingly, an object of the present invention is to solve the problems of conventional techniques as described above, and provide an oral device for treatment of sleep disordered breathing which can prevent the sleep disordered breathing such as an obstructive sleep apnea syndrome that may occur during sleep, enables a patient to more comfortably wear, and can minimize the discomfort in a state of wearing.

### Technical Solution

According to an aspect of the present invention for achieving the above object, there is provided an oral device for treatment of sleep disordered breathing that includes an upper body in which at least a part of upper teeth is housed; a lower body in which at least a part of the lower teeth is housed; at least one or more elastic members that connect the upper body and the lower body to provide the elastic force in a direction in which the upper body and the lower body come closer to each other; and a support jaw that protrudes downward from the rear end of the upper body to support the rear end of the lower body.

In the central portion of the upper body, a supporting portion made of resin ductile than the upper body and is in close contact with the front surface of the upper teeth may be provided.

Both sides of the supporting portion may be connected to the upper body by a connecting member.

The upper body and lower body may include at least one upper hooks and lower hooks to which both ends of the elastic member are caught, respectively.

The upper hook and the lower hook may be provided to form a pair each other.

Both ends of the elastic member may be caught to the upper hook and the lower hook to be inclined so that the lower end is located behind the upper end.

The lower body may be connected to the upper body and the elastic member, in a state of being moved about 60 to 80% of the maximum forward movement distance. The oral device for treatment of sleep disordered breathing according to any one of claims 1 to 6, wherein the lower body is connected to the upper body and the elastic member, in a state of being moved about 60 to 80% of the maximum forward movement distance.

### Advantageous Effects

According to the present invention, it is possible to prevent the sleep disordered breathing such as an obstructive sleep apnea syndrome that may occur during sleep, enable a patient to more comfortably wear, and minimize the discomfort in a state of wearing.

### DESCRIPTION OF DRAWINGS

FIG. 1 is a perspective view illustrating a side surface of an oral device for treatment of sleep disordered breathing according to an embodiment of the present invention.

FIG. 2 is a perspective view illustrating a front of the oral device for treatment of sleep disordered breathing according to an embodiment of the present invention.

### BEST MODE

Hereinafter, embodiments of the invention will be described in detail with reference to the accompanying drawings. The drawings are attached hereto to help explain exemplary embodiments of the invention, and the present invention is not limited to the drawings and embodiments. In the drawings, some elements may be exaggerated, reduced in size, or omitted for clarity or conciseness.

The present invention can make various changes and can have several embodiments, and specific embodiments will be illustrated in the drawings and described in detail in the detailed description. However, this is not intended to limit specific embodiments of the present invention, and should be understood to include all changes, equivalents and substitutes that come within the spirit and scope of the present invention.

Further, terms used in this application are simply used for the purpose of describing the particular embodiments and are not intended to limit the present invention. Singular expressions include plural expressions, unless they are meant in a clearly different manner in the context. In this application, terms such as “includes” or “has” should be understood to specify the presence of features, numbers, steps, operations, constituent elements, components or a combination thereof described in the specification, rather than excluding the presence or additional possibility of one or other features, numbers, steps, operations, constituent elements, components or a combination thereof in advance.

Hereinafter, an embodiment of an oral device for treatment of sleep disordered breathing according to the present invention will be described in detail with reference to the accompanying drawings.

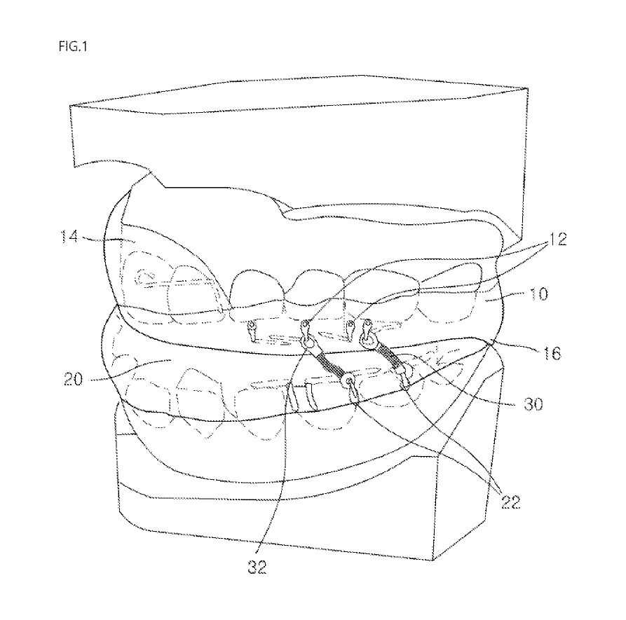

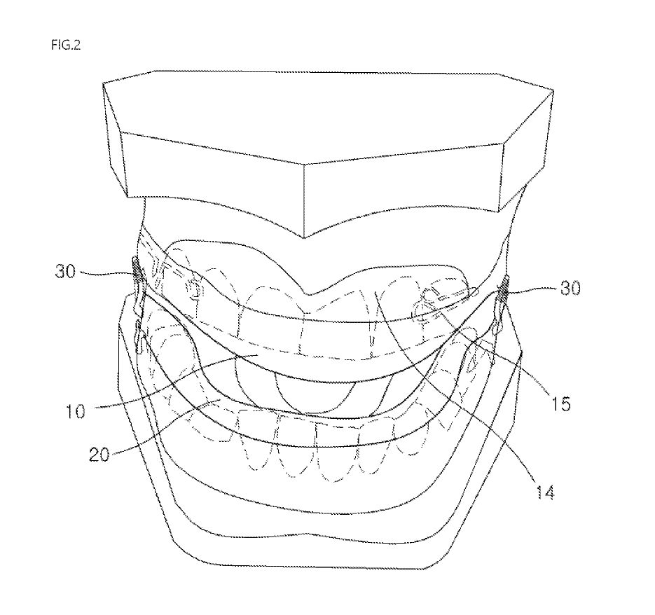

FIG. 1 is a perspective view illustrating a side surface of an oral device for treatment of sleep disordered breathing according to an embodiment of the present invention, and FIG. 2 is a perspective view illustrating a front of the oral device for treatment of sleep disordered breathing according to an embodiment of the present invention.

According to the illustrated configuration, the device according to the present invention may include an upper body in which at least a part of upper teeth is housed; a lower body 20 in which at least a part of the lower teeth is housed; at least one or more elastic members 30 that connect the upper body 10 and the lower body 20 to provide the elastic force in a direction in which the upper body 10 and the lower body 20 come closer to each other; and a support jaw 16 that protrudes downward from the rear end of the upper body 10 to support the rear end of the lower body 20.

The upper body 10 and the lower body 20 are made of resin and can be formed of a transparent or translucent material. In particular, the upper body 10 and the lower body 20 are made of hard resin to protect the teeth and masticatory muscle and the temporomandibular joint by biting, and the upper body 10 and the lower body 20 are capable of adjustment of the posterior occlusal surface, and can provide a more stable occlusion. For example, when the resins of the upper teeth, the lower teeth and molar abut against one another and when the mouth is closed, the contact portion is adjusted to evenly distribute the force. Therefore, the oral device can also be applied to the odontoprisis patient.

The upper body 10 is a portion in which the upper teeth of the wearer is housed, all or a part of the upper teeth can be housed therein. Similarly, the lower body 20 is a portion in which the lower teeth of the wearer is housed, and all or a part of the lower teeth can be housed therein. The upper body 10 and the lower body 20 can be differently manufactured depending on the structure of the teeth of the wearer.

At least one or more upper hooks 12 are provided on the outer surface of the upper body 10. The upper hooks 12 are portions to which the elastic member 30 is caught, and may have a shape in which the tip portion is bent upwards to catch the elastic member 30. That is, the upper hook 12 is made of material of the metal or alloy unlike the resin of the upper body 10, one end portion is mounted on the upper body 10 and may be formed integrally with the upper body 10. A plurality of upper hooks 12 may be provided an outer surface of the upper body 10 at predetermined intervals, and by providing the plurality of upper hooks 12 in this way, it is possible to provide the elastic member 30 in a desired part depending on the users of convenience.

Further, in the central portion of the upper body 10, a supporting portion 14 made of resin ductile than the upper body 10 is provided. The supporting portion 14 is in close contact with the front surface of the upper teeth to serve to buffer the force applied to the rear on the upper body 10. When a wearer moves the lower jaw backward while wearing the oral device, the upper body 10 connected to the lower body 20 also undergo a force of moving backward. At this time, since the supporting portion 14 is in close contact with the front surfaces of the upper teeth, it disperses and buffers the force applied to the upper body 10. To this end, the supporting portion 14 may have a sufficient size enough to cover the front surfaces of the upper teeth, and for the sake of buffering, the supporting portion 14 may be made of ductile resin softer than the upper body 10 as described above.

The supporting portion 14 is integrally formed with the upper body 10 and only its material can be formed in a different way as described above. Moreover, as shown in FIG. 1, the supporting portion 14 may be connected to the central portion of the upper body 10 by another connector 15. At this time, the connector 15 connects both sides of the supporting portion 14 to the upper body 10.

On the other hand, a supporting jaw 16 is formed to protrude downward at the rear end of the upper body 10. The supporting jaw 16 protrudes from the rear end of the upper body 10 to serve to support the rear end of the lower body from being pushed backward. The sleep disordered breathing occurs due to a phenomenon in which the respiratory tracts are blocked during sleep, and in order to treat the sleep disordered breathing, it is necessary to push the lower jaw forward in order to open the respiratory tract during sleep, and it is necessary to prevent the lower jaw from being pushed backward. For this reason, in this embodiment, in order to prevent the lower jaw from being pushed backward, the support jaw 16 is provided at the rear end of the upper body 10.

Further, the support jaw 16 may adopt any shape as long as it is intended to support the rear end of the lower body 20, and as shown in FIG. 1, the support jaw 16 can be integrally formed with the upper body 10 by machining the rear end of the upper body 10.

Next, a lower hook 22 corresponding to the upper hook is provided on the outer surface of the lower body 20. The plurality of lower hooks 22 may be provided at a predetermined interval, similarly to the upper hooks 12. The lower hook 22 may form a pair with the upper hook 12, and a plurality of the upper hooks 12 and the lower hook 22 forming the pair may be provided.

On the other hand, both ends of the elastic member 30 are caught to the lower hook 22 and the upper hook 12. The both ends of the elastic member 30 are supported by the upper hook 12 and the lower hook 22 to provide an elastic force in a direction in which upper body 10 and lower body approach each other. In the present embodiment, by connecting the upper body 10 and the lower body 20 via the elastic member 30 in this way, a wearer can comfortably move the upper and lower jaws in the vertical direction as well as the lateral direction, while wearing the oral device. In other words, the lower body 20 and the upper body 10 maintain a state of being connected to each other by the elastic member 30, and can relatively move, while offering an elastic force of the elastic member 30. In the drawings, although a coil spring is described as an example of the elastic member 30, it is not limited thereto, and various members such as rubber having elasticity can be used. For reference, reference numeral 32 is a connection hook formed at both ends of the elastic member 30, and is a portion that is substantially caught by the upper 12 and the lower hook 22.

Further, in the present embodiment, the elastic member 30 can be caught by the hooks 12, 22 such that lower end is inclined so as to be positioned behind the upper end. In the case of patients with sleep disordered breathing, as mentioned above, it is important to secure the respiratory tracts so that the lower jaw moves forward as mentioned above. Accordingly, it is necessary to restrict the lower jaw from moving backward by utilizing the elastic force of the elastic member 30, and when the elastic member 30 is caught by the hooks 12, 22 such that lower end is inclined so as to be positioned behind the upper end, it is possible to effectively restrict the lower body 20, that is, the lower jaw from moving backward. At this time, the forward movement of the lower jaw is, of course, acceptable.

Then, as described above, since the plurality of the lower hooks 22 and the upper hooks 12 are configured to form a couple, it is possible to mount the elastic member 30 at a desired position depending on the oral structure and convenience of the wearer.

Additionally, the elastic member 30 can connect the upper body 10 and lower body 20, in a state in which 60-80% (70% is the most suitable) of the maximum forward movement distance of the lower body 20, that is, the maximum distance that the wearer can move the lower jaw forward as lower body 20. This is to prevent the moving backward while the lower body 20 is moved fully forward. Thus, when the elastic member 30 is connected, while the lower body 20 has moved forward, the elastic member 30 can be restricted from being moved backward, since the lower body 20 receives the elastic force in the first connection state.

Hereinafter, a course of treatment by the oral device for treatment of sleep disordered breathing according to the present invention having the configuration as described above will be described in detail.

Patients presenting with snoring and obstructive sleep apnea syndrome need to wear the oral device on the teeth, before sleep. At this time, the oral device can be fabricated to fit the mouth structure of the wearer in advance. In particular, in this embodiment, the elastic member 30 is mounted in the state in which the wearer moves the lower jaw about 60 to 80% of the maximum forward movement distance (70% is most suitable). The elastic member 30 restricts the lower body 20 from moving backward, by being caught to the hooks 12, 22 such that lower end is positioned behind the upper end.

When a wearer sleeps with such an assembled oral device worn, since the upper body 10 and the lower body 20 are connected to each other by the elastic member 30, they can freely move in the vertical lateral and the lateral direction. Thus, it is possible to minimize a phenomenon in which the wearer feels discomfort, while wearing the oral device.

Further, when the wearer attempts to move the lower jaw backward, the movement is primarily restricted by the elastic force of the elastic member 30, and the rear end of the lower body 20 can be secondarily supported by the supporting jaw 16. Accordingly, it is possible to prevent the therapeutic effect from decreasing due to the situation that the wearer moves the lower jaw backward.

Further, when the wearer attempts to move the lower jaw backward, the supporting portion 14 may buffer the external force that is applied to the upper teeth by the upper body 10.

While the embodiments of the present invention have been described in detail, the scope of the present invention is not limited to this, using the basic concepts of the present invention that are defined in the following claims or modifications of those skilled in the art also belong to the scope of the present invention.

1. An oral device for treatment of sleep disordered breathing comprising:
an upper body in which at least a part of upper teeth is housed;
a lower body in which at least a part of the lower teeth is housed;
at least one or more elastic members that connect the upper body and the lower body to provide the elastic force in a direction in which the upper body and the lower body come closer to each other; and
a support jaw that protrudes downward from the rear end of the upper body to support the rear end of the lower body.

2. The oral device for treatment of sleep disordered breathing according to claim 1, wherein, in the central portion of the upper body, a supporting portion made of resin ductile than the upper body and is in close contact with the front surface of the upper teeth is provided.

3. The oral device for treatment of sleep disordered breathing according to claim 2, wherein both sides of the supporting portion are connected to the upper body by a connecting member.

4. The oral device for treatment of sleep disordered breathing according to claim 2, wherein the upper body and lower body comprise at least one upper hooks and lower hooks to which both ends of the elastic member are caught, respectively.

5. The oral device for treatment of sleep disordered breathing according to claim 4, wherein the upper hook and the lower hook are provided to form a pair each other.

6. The oral device for treatment of sleep disordered breathing according to claim 4, wherein both ends of the elastic member are caught to the upper hook and the lower hook to be inclined so that the lower end is located behind the upper end.

7. The oral device for treatment of sleep disordered breathing according to claim 1, wherein the lower body is connected to the upper body and the elastic member, in a state of being moved about 60 to 80% of the maximum forward movement distance.
